(19) United States
(12) Patent Application Publication
(10) Pub. No.: US 2008/0021498 A1
(43) Pub. Date: Jan. 24, 2008

(54) Disposable acupressure relief band

(76) Inventor: Joseph Di Lustro, Tinton Falls, NJ (US)

Correspondence Address: Charles I. Brodsky, 2 Bucks Lane, Marlboro, NJ, 07746, US

(21) Appl. No.: 11/490,274

(22) Filed: Jul. 21, 2006

(51) Int. Cl. A61B 17/00

(52) U.S. Cl. 606/204

(57) ABSTRACT

A disposable adhesive acupressure band for nausea relief in humans, dogs & cats, including a raised contact button on an adhesive lamination positioned over an acupressure point after peel off of a top liner centered about a cutout on the band through which the contact button extends from a bottom substrate.

### CROSS-REFERENCE TO RELATED APPLICATIONS

NONE.

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

NOT APPLICABLE

### BACKGROUND OF THE INVENTION

1. Field of the Invention

This invention relates to acupressure sensory relief bands, in general, and to a disposable acupressure band for use with people, dogs and cats, in particular.

2. Description of the Related Art

As is understood in the medical and surgical community, approximately 50 percent of all surgical procedures are impacted by postoperative nausea and vomiting (PONV). The usual procedure to deal with this is for an anesthesiologist to provide an anti-sickness drug before an anesthesia is administered—and then, to provide a further drug when the patient comes out of the surgery (i.e., “Drug Rescue Therapy”). Unfortunately, even this does not always work. As is also understood, similar administrations of drugs are made to prevent chemo induced nausea and vomiting (CINV), but with comparable less-than-optimum results.

Recent research in the field report a degree of success in the use of acupressure to alleviate postoperative nausea and vomiting, especially in ambulatory surgery. Likewise, research has indicated a degree of success of acupressure in preventing nausea and vomiting in chemotherapy treatments, beyond the use of antiemetic drugs. In each instance the use of acupressure reduced the incidence of nausea and vomiting.

Although acupressure wrist bands have been utilized in the past for the casual prevention of nausea (as for travel motion sickness and early pregnancy nausea prevention), they typically sell for about $10.00-$12.00 per pair, one for each hand. As will become clear from the following description, however, the acupressure relief band of the present invention obviates the hassle accompanying a medical patient's attempted use of such bands, along with a concomitant reduction in cost when tailored for use by a medical and surgical staff. As will also become clear, the acupressure relief band of the invention is at the same time disposable, hypoallergenic, skin contact friendly—and dimensionable for use both by people, and also by dogs and cats who frequently exhibit the same symptoms of motion sickness and of nausea and vomiting after surgery.

### SUMMARY OF THE INVENTION

Intended for positioning at the pericardium 6 (P6 meridian point on the wrist) or at the Pe6 meridian point on the foreleg of the dog or cat, the disposable acupressure relief band of the invention includes a band of prescribed length and width having a top liner over a bottom substrate. An adhesive lamination is provided on a top surface of the substrate, and a cutout is provided through the top liner to the substrate across the width of the band. To apply the acupressure, a raised contact button is included on the lamination, extending up through the cutout. In a preferred construction, the raised contact button is of a circular configuration, and the top liner is peelable away from the substrate at opposite sides of the cutout.

As will be appreciated by those knowledgeable in the art, a pair of such bands are utilized together—one on each wrist of a human, for example, or one on each foreleg above the carpal or wrist bone of the dog or cat. An acupressure relief band package including two of these bands can thus be provided within a pouch, according to the invention, with instructions for use printed directly on the pouch. One advantage of this will be seen to be that several such pouches could be removed from a box in which they come, to be taken to different operating rooms from a single supply center—without having to worry about the instructions for use only being printed on the box; this way, each member of the professional staff who emplaces or removes the band on or from a patient has his/her own set of instructions available to them.

As will become clear from the following description, a single embodiment of the invention is described for use on a human, or for use on a dog or cat. As will be seen, the difference lies in the application of the acupressure relief band, but having the same location of the cutout through which the contact button extends.

### BRIEF DESCRIPTION OF THE DRAWINGS

These and other features of the present invention will be more clearly understood from a consideration of the following description, taken in connection with the accompanying drawings, in which:

FIGS. 1 and 2 are helpful in an understanding of the use of the disposable acupressure relief band of the invention;

FIGS. 3a and 3b are top and side views of the top liner and bottom substrate of the relief band; and

FIG. 4 is an illustration of the raised contact button which extends through the cutout according to the invention.

### DETAILED DESCRIPTION OF THE INVENTION

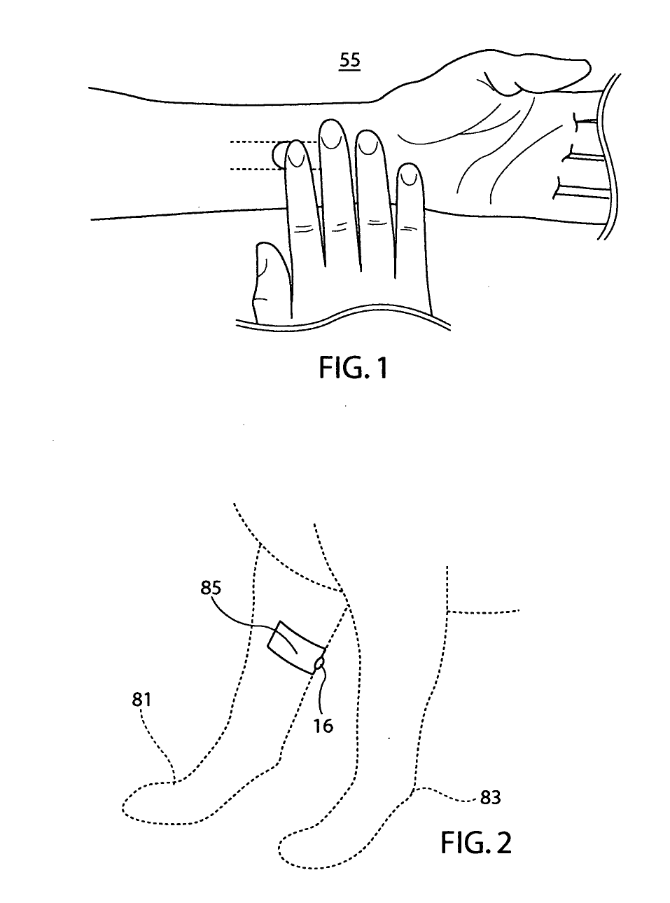

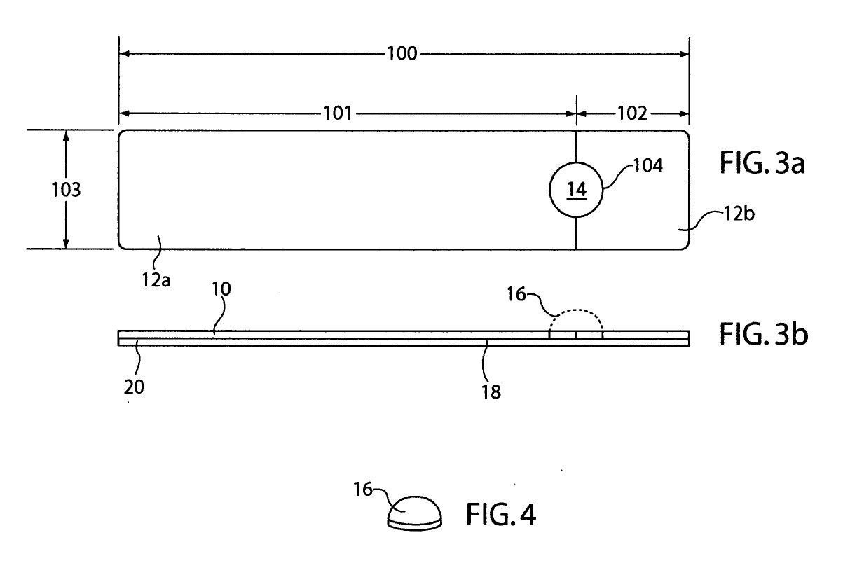

Referring to FIGS. 1, 3a, 3b and 4, the positioning of the acupressure relief band of the invention for use on both wrists of a human follows the placing of the first three fingers on the wrist crease on its inside, where the P6 pressure point is situated between the two flex tendons 55 under the index finger. The top liner 10 of the relief band is then peeled off on one side 12a of the cutout 14, then off the other side 12b. The contact button 16 of the band is then placed face down on the P6 pressure point on each wrist. The adhesive lamination 18 on a top surface of the bottom substrate 20 is then applied across the forearm area to maintain comfortable pressure on the P6 point throughout. The cutout 14 may be centrally placed along the band and from top-to-bottom, but offset from side-to-side.

In use by the professional medical staff in putting on and then taking off the band after use, a compress pressure is impacted to the contact button 16 on each wrist before the emetic stimulus or chemo medication is initiated. Investigation has shown that the P6 point is typically activated within 5 minutes from the time the pressure is compressed on both contact buttons. After the procedure is completed, the relief band 59 is simply peeled away from the wrist and removed—heightened by the adhesive lamination being selected as hypoallergenic and skin contact friendly.

As will be appreciated by those skilled in the art, in dimensioning the relief band of the invention, it is not necessary for the band to wrap completely around the user's wrist even one time. What is necessary is that the band be of sufficient width and the contact button of sufficient diameter (where the cutout 14 is circular) to overlap in providing sufficient pressure at the P6 pericardium point. In one construction of the disposable acupressure relief band for use by a human, the following dimensions were found useful:

Length 100 . . . 5.0 inches

Length 101 . . . 4.0 inches

Length 102 . . . 1.0 inches

Width 103 . . . 1.0 inches

Cutout Diameter 104 . . . 0.5 inches

A hypoallergenic pressure sensitive adhesive identified as product number 1527L from the 3M Company has worked quite well, along with a hemispherical contact button from Action Fabricators Inc. of product number 3002 designation. Such contact button, measuring 0.44 inches in diameter and 0.20 inches in height worked quite well with the 3M adhesive strip.

Testing has shown that an acupressure nausea protector of this configuration provided very satisfactory results for more than 24 hours, and with or without the use of antisickness drugs. As will be apparent, besides being used to prevent postoperative nausea and vomiting, and chemo induced nausea and vomiting in a medical procedure environment, the acupressure relief band of the invention could also be used as a preventer of travel motion sickness and of early pregnancy nausea as well.

When packaged with two such bands in a heat sealed pouch, the pouch can be imprinted with the following directions:

Nausea Relief Instructions for Adult Men and Women
Intended Use: For Men and Women for the relief of nausea symptoms Related to Surgery, travel, chemotherapy and morning sickness.
1. Locate pressure point on each wrist, using the three middle fingers of your other hand and placing them centrally on the inside of the wrist, starting with the first crease between the two central tendons. The point is located under the index finger.
2. Once located, massage or press down on the point area for 30 seconds to activate the relief.
3. Place band with the contact button face down over point.
4. Remove liners from adhesive and apply around the wrist.
5. Repeat with other wrist.

With a pouch packaging two such bands of the invention for use with dogs and cats, the instructions printed on the back of the pouch may be as follows:

Nausea Relief Instructions for Dogs & Cats
Intended Use: Relief of nausea symptoms related to surgery and travel.
1. Locate pressure point on each foreleg, using your index finger and place it centrally on the deepest part of the back of the foreleg area, above the carpal or wrist bone, between the two central tendons.
2. Once located, massage the point area for 30 seconds to activate relief.
3. Place band with the contact button face down over point.
4. Remove liners from adhesive and wrap comfortably around leg area.
5. Repeat with other foreleg.

### FIG. 2 shows the forelegs 81 of the dog or cat, the wrist bone 83, and the contact button 16 and band 85 in place.

The same dimensions used for the disposable acupressure relief band for humans could be used for the relief band for these pets as well. The configuration of the band can then suit both applications.

The instructions for human use and for dog and cat use as set out above could be printed on an instruction card for the heat sealed pouch packaging the two disposable acupressure relief bands employed for the acupressure nausea protection of the invention.

While there have been described what are considered to be preferred embodiments of the present invention, it will be readily appreciated by those skilled in the art that modifications can be made without departing from the scope of the teachings herein of a hypoallergenic, skin contact friendly disposable band which produces continuous acupressure stimulation for nausea protection. For at least such reason, therefore, resort should be had to the claims appended hereto for a true understanding of the scope of the invention.

I claim:

1. A disposable acupressure relief band comprising:
a band of prescribed length and width having a top liner over a bottom substrate;
an adhesive lamination on a top surface of said substrate;
a cutout through said top liner to said bottom substrate across the width of said band; and
a raised contact button on said lamination extending through said cutout.

2. The disposable relief band of claim 1 wherein said adhesive lamination is hypoallergenic.

3. The disposable relief band of claim 1 wherein said adhesive lamination is a skin contact lamination.

4. The disposable relief band of claim 1 wherein said top liner is peelable away from said bottom substrate at opposite sides of said cutout.

5. The disposable relief band of claim 1 wherein said cutout is offset positioned along the length of said band.

6. The disposable relief band of claim 5 wherein said cutout is offset positioned one fifth of the length in from one end of said band.

7. The disposable relief band of claim 5 wherein said cutout is centrally positioned from top and bottom of said band.

8. The disposable relief band of claim 5 wherein said cutout is offset positioned one fifth of the length in from one end of said band, and centrally positioned from top and bottom of said band.

9. The disposable relief band of claim 8 wherein said band is of a 5.0 inch length and 1.0 inch width, and wherein said cut through is circular of a diameter of 0.50 inches.

10. A disposable acupressure relief band package comprising:
first and second bands, each of prescribed length and width and each having a top liner over a bottom substrate;
an adhesive lamination on a top surface of each bottom substrate;
a cutout through each top liner to each bottom substrate across the width of each band;
and a raised contact button on each lamination extending through their respective cutouts; and
with both of said bands included within a heat sealed pouch.

11. The disposable relief band package of claim 10 wherein said package is printed with instructions for use on one of men & women, dogs & cats.

12. A disposable acupressure relief band box comprising:
a plurality of heat sealed pouches, each printed with instructions for use on men & women, or dogs & cats, and each pouch including first and second bands, each of prescribed length and width and each having a top liner over a bottom substrate;
an adhesive lamination on a top surface of each bottom substrate;
a cutout through each top liner to each bottom substrate across the width of each band;
and a raised contact button on each lamination extending through their respective cutouts.
